(19) United States
(12) Patent Application Publication
(10) Pub. No.: US 2004/0092861 A1
(43) Pub. Date: May 13, 2004

(54) Methods and systems for performing vitrectomy with continuous perfluorocarbon infusion

(75) Inventor: Hugo Quiroz-Mercado, Mexico City (MX)

Correspondence Address: Robert D. Buyan, Stout, Uxa, Buyan & Mullins, LLP, Suite 300, 4 Venture, Irvine, CA, 92618, US

(73) Assignee: S.K. Pharmaceuticals, Inc., San Juan Capistrano, CA

(21) Appl. No.: 10/392,273

(22) Filed: Mar. 19, 2003

Related U.S. Application Data
(60) Provisional application No. 60/365,881, filed on Mar. 19, 2002.

(51) Int. Cl. A61B 17/20

(52) U.S. Cl. 604/22

(57) ABSTRACT

A method and system for continuous infusion of a dense fluid (e.g., a perfluorocarbon) during performance of a vitrectomy procedure. A vitrectomy cutter device is used to remove vitreous humor from the posterior chamber of the eye and a continuous flow of the dense fluid is infused in the posterior chamber concurrently with the removal of the vitreous through the vitrectomy cutter device. In a recirculating embodiment, a re-circulation loop is formed whereby particles of vitreous removed from the eye are filtered out and the dense fluid is then re-circulated back into the posterior chamber of the eye. The dense fluid may be oxygenated before entering the eye.

### RELATED APPLICATION

This application claims the benefit of U.S. Provisional Application Serial No. 60/365,881, filed Mar. 19, 2002, the entirety of which is expressly incorporated herein by reference.

### FIELD OF THE INVENTION

The present invention generally relates to ophthalmic surgery. More particularly, the invention relates to methods and systems for performing vitrectomy procedures with a continuous flow of a dense fluid, such as a perfluorocarbon fluid, into the posterior chamber of the eye.

### BACKGROUND OF THE INVENTION

The vitreous humor is a gel-like substance, consisting primarily of water, that fills the posterior chamber of the eye, behind the crystalline lens. Its function is to give shape to the eye, transmit light, and form a semi-solid support for the retina In some patients suffering from diabetic retinopathy, vitreous hemorrhage, hemolytic glaucoma, central vein occlusion, macular holes or tears and other retinal disorders, it is sometimes desirable to remove the vitreous humor in a procedure known as a vitrectomy. The vitrectomy procedure typically begins with the creation of three small incisions through the sclera and pars plana, into the posterior chamber wherein the vitreous is contained. An illuminating fiberoptic light pipe is inserted through one incision and an infusion tube is inserted through the second incision. A vitreous cutter/aspirator probe is inserted through the third incision. The vitreous cutter/aspirator probe is then used to remove the vitreous from the posterior chamber while a gravity-fed flow of make-up fluid such as basic salt solution enters the posterior chamber through the infusion tube, thereby maintaining relatively constant pressure within the posterior cavity. Such removal of the vitreous can relieve traction on areas of the retina that are damaged or diseased. Also, in cases where the vitreous gel has become clouded with hemorrhagic blood or where other opacities are present, removal of the vitreous can clear the surgeon's view of the retina. With such improved ability to view the retina, the surgeon may in some cases be able to perform various retinal procedures such as endolaser photocoagulation and/or endocryopexy, using special probes. Also, in some cases where a portion of the retina has become detached, performance of the vitrectomy can allow subretinal fluid to drain which alone may hydraulically reattach the retina. At the end of the operation, the surgeon closes the pars plana incisions and may fill the vitreous cavity with an air or gas bubble to tamponade the retina internally.

In some cases, such as those where there is some tractional and/or rhegmatogenous retinal detachment, it may be desirable to introduce a dense fluid, such as a perflorocarbon liquids (e.g., perfluoro-n-octane, perfluorodecaline) or certain oils (e.g., silicon oil) to provide temporary tamponade of the affected retina, to effect retinal flattening, to effect transscleral fixation of posterior chamber lenses, to facilitate endophotocoagulation, or for other reasons. In some cases, the dense fluid may be at the end of the procedure. In other cases the dense fluid may be allowed to remain in the eye to provide post-surgical benefit.

U.S. Pat. No. 4,490,351 (Clark, Jr.) describes the injection of liquid perfluorocarbons and substituted derivatives thereof (e.g., perfluorooctylbromide (PFOB), perfluoro 1-methyldecalin (PP9), and perfluoro 1,3-dimethyladamantane and perfluorotrimethylbicyclo[3.3.1.]nonane mixtures (DAWN)) as fluid substitutes for the vitreous humor. The entire disclosure of U.S. Pat. No. 4,490,351 (Clark, Jr.) is expressly incorporated herein by reference.

Although the injection of perfluorocarbon fluids into the eye to replace the vitreous has proven to be a viable and beneficial procedure, there remains a need in the art for the development of improved methods and systems for introducing the perfluorocarbon fluids into the posterior chamber of the eye.

### SUMMARY OF THE INVENTION

The present invention provides a system and methods for performing surgical procedures, particularly ophthalmic procedures, especially vitrectomy procedures.

In a first aspect, the invention relates to a system for treating a patient during an ophthalmic procedure, comprising delivery apparatus for delivering a fluid to an ophthalmic surgical site, and removal apparatus for removing the fluid from the surgical site. Optionally, the system may also include apparatus for recycling or returning the fluid back to the surgical site. The fluid delivery apparatus may include a fluid reservoir containing a dense fluid, and means, such as ducting, a conduit or tube, for introducing the dense fluid into the surgical site. The removal apparatus may include apparatus, such as a conduit, tube or ducting, for withdrawing the fluid from the surgical site. When this invention is applied to vitrectomy surgery, a fluid supply tube is inserted into the posterior chamber of the eye concurrently with a vitrectomy device which cuts and aspirates the vitreous humor. As the vitrectomy device removes vitreous humor from the eye, a flow of dense fluid enters the posterior chamber through the fluid supply tube. Such dense fluid may optionally be oxygenated and/or filtered before it enters the eye. Such dense fluid may provide oxygen to the retina and other structures of the eye and may serve to tamponade the retina and/or any bleeding sites within the posterior chamber. Also, such dense fluid may be substantially clear such that the surgeon may view the procedure or anatomical structures within the eye, through such fluid.

In some embodiments of the invention, the dense fluid that is removed form the surgical site may be recycled back into the surgical site. In this regard, the removal apparatus may also includes a peristaltic pump for circulating the fluid throughout the system. In recirculating and/or non-recirculating embodiments of the invention, the system may optionally include a reservoir for collecting the fluid, a filtration apparatus for filtering or removing particulate matter from the fluid and/or an oxygenator for oxygenating the fluid.

Further in accordance with the invention, there is provided a method and system wherein the dense fluid is a dense or heavy fluid having a density greater than water (n_{D}²⁰), preferably an n_{D}²⁰ 1.05, 1.1, 1.2, 1.3, 1.4 or greater. A density of about 1.1 to about 1.3 or 1.4 is particularly suitable. Fluorinated fluids, particularly perfluorocarbon liquids (PFCLs) are presently preferred.

In a second aspect, the invention comprises a method for treating a surgical site during a surgical procedure, including the steps of delivering a fluid to the surgical site, removing the fluid from the surgical site, and redelivering the fluid to the surgical site. In an especially preferred embodiment, the surgical site is an eye, and the fluid is perfluorinated fluid. The step of delivering the fluid to the patient's eye preferably comprises providing a perfluorinated fluid delivery system, wherein the perfluorinated fluid delivery system includes a reservoir containing PFCL, and ducting or other supply means leading from the reservoir to the eye. The step of removing the fluid preferably includes providing a vitrectomy probe, and the step of redelivering the fluid preferably includes ducting, a peristaltic pump, filtration means, and oxygenation means.

Further aspects and advantages of the invention will become apparent to persons of skill in the art upon reading and understanding of the detailed descriptions of the preferred embodiments set forth herebelow.

### BRIEF DESCRIPTION OF THE DRAWINGS

FIG. 1 is a schematic showing of an apparatus for aspirating and replacing intravitreal fluid according to the present invention, along with a lateral cross-sectional view of a human eye;

FIG. 2 is a diagrammatic view showing a method of aspirating and replacing intravitreal fluid according to the present invention, along with a lateral cross-sectional view of a human eye; and

FIG. 3 shows a preferred fluid reservoir in accordance with the present invention.

### DETAILED DESCRIPTION AND EXAMPLES

The following detailed description, and the accompanying drawings to which it refers, are provided describing and illustrating certain examples or specific embodiments of the invention only and not for the purpose of exhaustively describing all possible embodiments and examples of the invention. Thus, this detailed description does not in any way limit the scope of the inventions claimed in this patent application or in any patent(s) issuing form this or any related application.

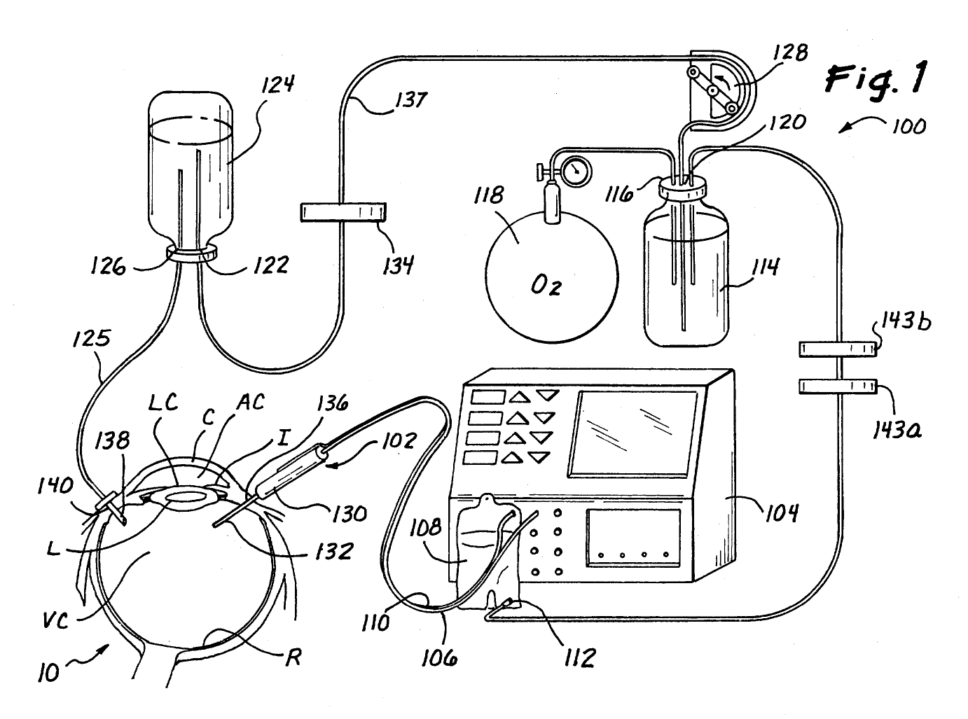

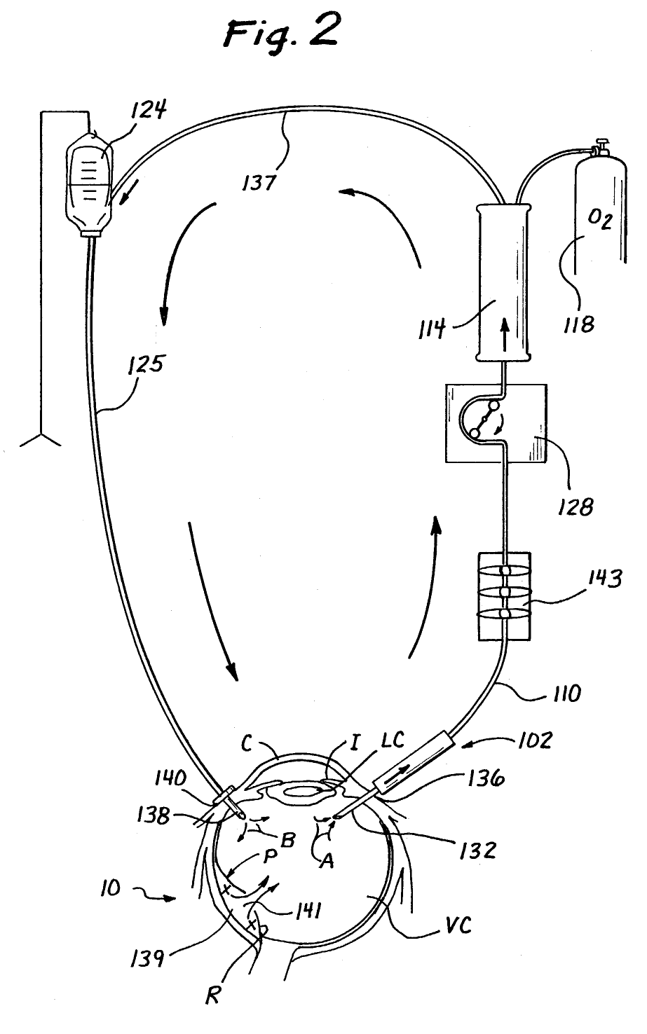

FIGS. 1 and 2 of this application contain illustrations of the human eye 10. The anatomical structures of the eye, shown in these figures, are labeled in accordance with the following legend.

| | |
|---|---|
| Cornea | C |
| Anterior Chamber | AC |
| Iris | I |
| Lens Capsule | LC |
| Lens | L |
| Vitreous Chamber | VC |
| Retina | R |

As shown in FIGS. 1-2, a system 100 of the present invention generally comprises a removal device 102 coupled to a control console 104 by means of a drive cable 106, and to an upper portion of a collection bag 108 by a suction outlet tube 110. An outlet port 112 in a lower portion of the collection bag 108 is in turn coupled to an oxygenation bottle 114. The oxygenation bottle has an oxygen inlet port 116 for receiving oxygen from an oxygen source 118, and an outlet port 120 coupled to an inlet port 122 of a fluid reservoir 124. Fluid introduction means 125, such as tubing made from polypropylene or tetrafluoroethylene is coupled to an outlet port 126 of the fluid reservoir 122. A peristaltic pump 128 is provided for circulating fluid through the system.

The removal device 102 may comprise an aspiration device for removing and transporting fluid from the eye 10, and more specifically, may be a vitrectomy probe including a handpiece 130 and hollow needle 132, with a blade or other cutting device (not shown) mounted for reciprocation within the needle. Suitable probes, as well as their associated control modules, are commercially available, for instance, under the trade names Accurus® and InnoVit® from Alcon Labs of Fort Worth, Tex. In addition, examples of such probes are disclosed in U.S. Pat. No. 4,696,298 to Higgins et al. and U.S. Pat. No. 5,037,384 to Chang.

In a preferred embodiment of the invention, the fluid reservoir 124 contains a dense fluid, more specifically a fluid having a density (n_{D}²⁰) of greater than 1.0, and preferably, an n_{D}²⁰ of about 1.05, 1.1, 1.2, 1.3 1.4 or greater. A density of about 1.1 to about 1.3 or 1.4 is particularly suitable. Optimally, the fluid should also have a high affinity for oxygen, and higher superficial tension and lower viscosity than water, while its refractive index should be equivalent to that of water. Fluorinated liquids, and particularly perfluorocarbon liquids (PFCLs) have been found to be especially useful for the purposes of this invention. Especially suitable PFCLs include perfluorocarbon solvents having from about 3 to about 12 carbons, such as perfluorheptane or perfluoroctane (C₈F₁₈). Successful trials of the method according to the present invention were performed using perfluoroctane having a viscosity of 0.8 centistokes, a refractive index of 2.7, and specific gravity of 1.7. The perfluoroctane (commercially available from sources in the United States or Europe) was purified with a 0.2μ filter 134 (for instance, a 0.2μMillipore™ filter, commercially available from Millipore Corporation of Bedford, Mass.) and sterilized with ethylene oxide before use.

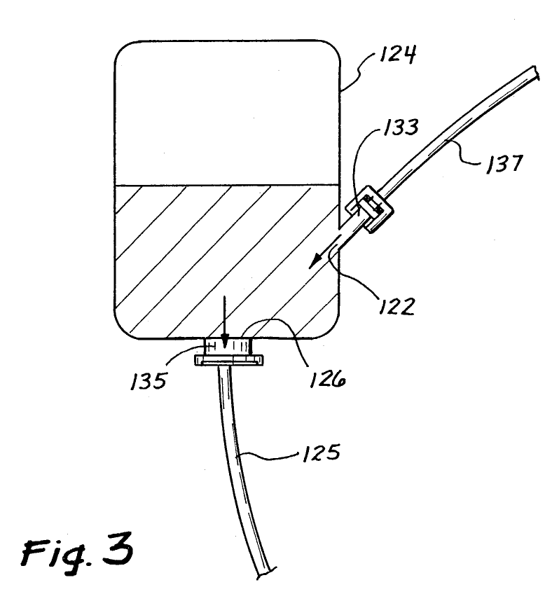

A preferred fluid reservoir 124, shown in FIG. 3, is preferably an infusion bottle formed of a sterilizable material such as glass. Preferably the bottle is transparent to enable medical personnel to view the volume of contained fluid. The outlet port 126 is located at the bottom of the reservoir 124, and the inlet port 122 located above the outlet port 126 to allow gravity-assisted flow. An airtight sealing neck 133 projects outwardly from the inlet port 122 to receive the tubing 137 leading from the oxygenation bottle 114, and a similar sealing neck 135 projects downwardly from the outlet port 135 to receive the fluid introduction means or tubing 125.

A method of treating a patient during an ophthalmic surgical procedure using the system 100 according to the present invention will now be described with reference to FIG. 2. For purposes of illustration, the method is shown being used on an eye 10 in which the retina R has become partially detached and torn, allowing a small amount of fluid 139 to accumulate below the tear 141. Removal of this subretinal fluid is essential, since its continued presence can result in complete retinal detachment and partial or total blindness. It is understood, however, that the method of the present invention is not restricted to use in treating the illustrated condition, but may be used in a wide variety of procedures including, but not limited to, procedures for managing or repairing retinal detachment with proliferative vitreoretinopathy, diabetic traction retinal detachments, giant tears, retinopathy of prematurity, endophthalmitis, posteriorly dislocated crystalline lens and intraocular foreign bodies, ocular trauma, and in the treatment of retinal ischemia, repositioning of dislocated intraocular lenses, surgical excision of subretinal membranes, and control of intraoperative hemorrhage.

Initially, a patient's eye 10 is prepared for surgery, for instance by administering retrobulbar, peribulbar or subjconjuctival anesthetic. A facial nerve block may also be used. The hollow needle or distal tip 132 of the vitrectomy probe 102 or similar aspiration device is inserted transconjunctivally through a first incision 136 in the pars plana of the eye, and a distal tip 138 of the fluid introduction means 125 is inserted transconjuctivally through a second incision 140 in the pars plana, preferably at a location 90° to 180° from the first incision 136. The cutting means within the probe 102 is then actuated to cut through fibers in the vitreous, and the peristaltic pump 128 is actuated to begin drawing the vitreous in the direction of arrows A through the suction outlet tube 110, at the same time delivering PCFL or other replacement fluid from the fluid reservoir 124 to enter the vitreous chamber VC in the direction of arrows B through fluid introduction means 125. The flow rate of PCFL entering the chamber VC is controlled such that it substantially equals the flow rate of vitreous humor leaving the chamber VC, resulting in a substantially constant volume of added fluid in the eye. The PCFL, being heavier than the vitreous, settles to the bottom of the eye, thus forcing the vitreous upward.

As the PCFL enters the vitreous chamber VC, it collects in the form of small bubbles within the vitreous. Eventually, these bubbles coalesce into a single large bubble, indicating that the vitreous has been completely removed. The bubble exerts a pressure P on the retina R, forcing the detached portion back against the choroidal tissues and squeezing the subretinal fluid 139 anteriorly outward through the tear 141 as shown by arrows X. Once the retina R is flattened and stabilized by the PCFL, it can be reattached to the choroidal tissues using laser photocoagulation and/or other conventional procedures.

Any PCFL in excess of what is needed to form the stabilizing bubble is continuously drawn out by the aspiration device 102, together with any remaining vitreous and other particles such as blood cells, pigment, bacteria and/or membrane debris, and is then deposited in collection bag 108 (shown in FIG. 1 only). Due to its high specific gravity, the PCFL settles at the bottom of the collection bag 108, while the lighter vitreous remains on top. The PCFL is then aspirated from the collection bag 108 through the outlet port 112, and passed through a series of filters 143 to remove debris. In the preferred embodiment of FIG. 1, the filters 143 include a 5.0μ Millipore™ filter 143a and a 0.22μ Millipore™ filter 143b, both commercially available from Millipore Corporation of Bedford, Mass.

After filtration, the PCFL is pumped through the oxygenation bottle 114 where it is oxygenated by bubbling, and then returned to the fluid reservoir 124, preferably via an additional filter 134 (shown in FIG. 1 only). The recycled fluid may then be returned to the eye through the fluid introduction means 125, thus forming a complete, closed cycle.

After the surgical procedure has been completed; the PCFL may be left in the vitreous chamber VC as a permanent substitute for the vitreous humor if desired, or a PCFL-BSS interchange may be performed to remove all PCFL from the vitreous chamber. Such an exchange, if necessary, can be accomplished quickly and easily by simply replacing fluid reservoir 124 with a reservoir containing BSS (balanced saline solution). Other subsequent procedures, such as air/fluid exchange and gas or silicon oil tamponade, may also be performed.

It is to be understood that the foregoing specification has been presented by way of illustration only, and not limitation. Numerous alterations, changes, modifications, variations and the like will occur to those skilled in the art in view of the above described preferred embodiments of the present invention. Accordingly, the present invention is to be understood as being limited only by the terms of the claims appended hereto.

The present invention provides a number of benefits. For example, the delivery of oxygenated PFCL or other oxygenated liquid to the retina during the vitrectomy procedure will maintain a well-oxygenated retina even in long lasting virectomy procedures. Also, the continuous infusion of PFCL or other dense fluid will provide tamponade on the retina as well as on any bleeding sites. Additionally, because PFCLs are not miscible with the vitreous, the infused PFCL may provide a clear fluid through which the surgeon may view structures of the eye and the vitrectomy procedure, even in cases where the vitreous is cloudy due to hemorrhage or other opacities.

Although exemplary embodiments of the invention have been shown and described, many changes, modifications and substitutions may be made by those having ordinary skill in the art without necessarily departing from the spirit and scope of this invention. For example, elements, components or attributes of one embodiment or example may be combined with or may replace elements, components or attributes of another embodiment or example to whatever extent is possible without causing the embodiment or example so modified to become unuseable for its intended purpose. Accordingly, it is intended that all such additions, deletions, modifications and variations be included within the scope of the following claims. Also, although several illustrative examples of means for practicing the invention are described above, these examples are by no means exhaustive of all possible means for practicing the invention. The scope of the invention should therefore be determined with reference to the appended claims, along with the full range of equivalents to which those clams are entitled.

1. A method for performing a vitrectomy in the eye of a human or veterinary patient, said method comprising the steps of:
A) forming at least first and second opening into the posterior chamber of the eye;
B) inserting a vitrectomy cutter/aspiration probe through the first opening;
C) inserting a fluid supply conduit through the second opening,
D) using the vitrectomy cutter/aspiration probe to remove at least a portion of the vitreous humor from the posterior chamber while allowing a dense fluid to flow into the posterior chamber through the fluid supply conduit:

2. A method according to claim 1 further comprising the steps of:
providing a filter device having an inlet port, an outlet port and a filtration element which will retain particles of vitreous while allowing the dense fluid to pass therethrough;
connecting the vitrectomy cutter/aspiration probe to the inlet port of the filter device; and
connecting the outlet port of the filter device to the fluid supply tube to form a re-circulation loop whereby a mixture of dense fluid and particles of vitreous will flow from the vitrectomy cutter/aspiration probe and into the filter device, the particles of vitreous will be retained by the filter element, dense fluid fill flow from the outlet port of the filter device through the fluid supply tube and back into the posterior chamber of the eye.

3. A method according to claim 1 further comprising:
oxygenating the dense fluid as it passes through the recirculation loop.

4. A method according to claim 3 wherein the step of oxygenating the dense fluid is carried out by bubbling oxygen or an oxygen-containing gas mixture through the dense fluid after it has passed through the filter device.

5. A method according to claim 1 wherein the dense fluid comprises a perfluorocarbon.

6. A method according to claim 5 wherein the perfluorocarbon is selected from the group consisting of: perfluoroheptane; perfluorooctane, perfluoro-n-octane; perfluorodecaline; perfluorooctylbromide (PFOB); perfluoro 1-methyidecalin (PP9); and perfluoro 1,3-dimethyladamantane and perfluorotrimethylbicyclo[3.3.1.]nonane mixtures (DAWN)

7. A method according to claim 1 wherein the filtration element of the filter device comprises a membrane having pores that are approximately 0.22 microns to 500 microns in size.

8. A method according to claim 1 wherein the filter device comprises a plurality of filter devices at a plurality of locations on the recirculation loop.

9. A method according to claim 1 wherein the filter device comprises a first filtration element having approximately 5.0 micron pores and a second filtration element having approximately 0.22 micron pores.

10. A method according to claim 1 further comprising the step of positioning a reservoir in the re-circulation loop for collection of the dense fluid.

11. A method according to claim 1 further comprising the step of providing a pump and using said pump to pump dense fluid though the recirculation loop.

12. A system for continuous infusion of a dense fluid during performance of a vitrectomy procedure in the eye of a human or veterinary patient, said system comprising:
A) a vitrectomy cutter/aspiration probe insertable through a first opening into the posterior chamber of the eye;
B) a fluid supply conduit insertable through a second opening into the posterior chamber of the eye; and
C) a source of dense fluid connected to the fluid supply tube such that as vitreous is removed from the posterior chamber dense fluid will enter the posterior chamber through the fluid supply tube.

13. A system according to claim 12 further comprising:
a filter device having an inlet port, an outlet port and a filtration element which will retain particles of vitreous while allowing the dense fluid to pass therethrough;
the vitrectomy cutter/aspiration probe being connected to the inlet port of the filter device; and
the outlet port of the filter device being connected to the fluid supply tube to form a re-circulation loop whereby a mixture of dense fluid and particles of vitreous will flow from the vitrectomy cutter/aspiration probe and into the filter device, the particles of vitreous will be retained by the filter element, dense fluid will flow from the outlet port of the filter device through the fluid supply tube and back into the posterior chamber of the eye.

14. A system according to claim 12 further comprising:
oxygenation apparatus for oxygenating the dense fluid as it passes through the re-circulation loop.

15. A system according to claim 14 wherein the oxygenation apparatus comprises apparatus for bubbling oxygen or an oxygen-containing gas mixture through the dense fluid after it has passed through the filter device.

16. A system according to claim 12 wherein the source of dense fluid comprises a source of a perfluorocarbon fluid.

17. A system according to claim 16 wherein the source of perfluorocarbon fluid contains a perfluorocarbon selected from the group consisting of: perfluoroheptane; perfluoro-n-octane; perfluorodecaline; perfluorooctylbromide (PFOB); perfluoro 1-methyldecalin (PP9); and perfluoro 1,3-dimethyladamantane and perfluorotrimethylbicyclo[3.3.1.]nonane mixtures (DAWN)

18. A system according to claim 12 wherein the filtration element of the filter device comprises a membrane having pores that are approximately 0.22 micron-500 microns in size.

19. A system according to claim 12 wherein the filter device comprises a plurality of filter devices positioned at a plurality of locations on the recirculation loop.

20. A system according to claim 12 wherein the filter device comprises a first filtration element having approximately 500 micron pores and a second filtration element having approximately 0.22 micron pores.

21. A system according to claim 12 further comprising a reservoir in the recirculation loop for collection of the dense fluid.

22. A system according to claim 12 further comprising a pump for pumping the dense fluid though the recirculation loop.
